(19) United States
(12) Patent Application Publication
(10) Pub. No.: US 2023/0404841 A1
(43) Pub. Date: Dec. 21, 2023

(54) BRUSH

(71) Applicants: Jcraft Co., Ltd., Osaka (JP); BELEGA Co., Ltd., Osaka (JP)

(72) Inventor: Hiroshi UENO, Osaka (JP)

(21) Appl. No.: 18/029,704

(22) PCT Filed: Oct. 1, 2021
(86) PCT No.: PCT/JP2021/036471

§ 371 Date: Mar. 31, 2023

(30) Foreign Application Priority Data
Oct. 16, 2020 (JP) 2020-174671

(51) Int. Cl. A61H 7/00; A61N 1/04

(52) U.S. Cl. CPC A61H 7/005; A61H 2205/021; A61H 2201/5058; A61N 1/0476

(57) ABSTRACT

A brush includes a brush body, electrodes provided at the tips of brush bristles, a sensor provided on the surface of the brush body on which the brush bristles are provided, and a controller configured to control the motions of the brush bristles on the basis of the detection output signal of the sensor. The sensor detects the conditions of the scalp, and the brush performs electric stimulation by applying a current of a specific frequency, or performs ion introduction/lead-out by applying a weak current, so as to be suitable for the conditions of the scalp of a target portion.

### TECHNICAL FIELD

The present invention relates to a brush capable of performing scalp gunk removal and scalp massage while applying an electric current to a human body.

### BACKGROUND ART

Each of Patent Documents 1 to 4 discloses an item configured to activate hair growth tissue and improve blood circulation, by stimulating the scalp, so as to enable hair growth and prevent hair loss, or improve the conditions on the scalp or the hair quality.

Patent Document 1 (Japanese Patent Application Laid-Open Publication No. 2020-62401) discloses a dryer which includes a blower configured to suck air from the outside and blow the air toward an air outlet, a heater configured to heat the air to be blown away, a conductive brush which is attached to the air outlet in the blowing direction and includes a plurality of comb-shaped pins or brush-shaped pins to be brought into contact with user's scalp, including at least one pin made of conductive material among the pins, and an electrification device, so that the dryer is capable of applying an electric current to user's scalp through an electrode part made of the conductive material.

Patent Document 2 (Japanese Patent Application Laid-Open Publication No. 2005-194640) discloses a cap type planar electrode structure for stimulating scalp which includes a pair of planar flexible electrodes for sending a weak electric current having an alternating waveform into a scalp. The planar electrodes have a pair of conductive faces, which are made of conductive cloths and separated into two regions through an insulating zone and whose at least surfaces are formed of silver, and are formed into a cap-like shape so that the conductive faces are placed on the scalp side.

Patent Document 3 (Japanese Patent Application Laid-Open Publication No. 2002-369887) discloses a scalp energizing brush which includes a plurality of brush electrodes and a brush body including a frame body in a horizontally long band shape curved to surround the cephalic region, where the plurality of electrodes are planted.

The plurality of brush electrodes are disposed so as to be elastically pressed and energized outwardly in the axial direction to the frame body. Brush electrode groups including the plurality of brush electrodes are adjacently arrayed from the center to the both sides of the curved frame body. On the both ends of the frame body, a pair of flexible band-like elastic supporting bodies are provided so as to press and clamp both temples.

Patent Document 4 (Japanese Patent Application Laid-Open Publication No. S52-105058) discloses a beauty device which includes a motor, a blower fan configured to be rotated by the motor, an eccentric shaft inserted eccentrically from the center of the rotary shaft driven by the motor, a crank rod connected to the both sides of the eccentric shaft, and a pair of adjusters connected to the crank rod via a link mechanism so as to reciprocate.

The devices disclosed in Patent Documents 1 to 4 are configured to promote hair growth by bloodstream improvement of a scalp, prevent hair loss and improve hair quality, by pressing, vibration, electric stimulation, and iontophoresis. In either of the devices, the mechanical or electrical output intensity is manually controlled, and is not appropriately changed according to scalp conditions or hair conditions.

In other words, either of the devices disclosed in Patent Documents 1 to 4 has the problems in which, although the conditions of the scalp and hair are not uniform across the entire head, for example, if the so-called strongly damaged area is supposed to be subjected to high (strong)output intensity 1, a user may use the device with a low (weak) level according to a preference of the user, and may physically feel less effect. In another example, although the subjected output intensity at an undamaged portion should be low (weak), a user may use the device with a high (strong) level according to a preference of the user, and may cause unnecessary damage.

### CITATION LIST

### Patent Literature

[Patent Document 1] Japanese Patent Application Laid-Open Publication No. 2020-62401
[Patent Document 2] Japanese Patent Application Laid-Open Publication No. 2005-194640
[Patent Document 3] Japanese Patent Application Laid-Open Publication No. 2002-369887
[Patent Document 4] Japanese Patent Application Laid-Open Publication No. S52-105058

### SUMMARY OF INVENTION

### Technical Problem

The present invention is to solve the problems with respect to prior arts including Patent Documents 1 to 4 described above, in which although the scalp conditions and hair conditions are not uniform in the entire cephalic region, the output intensity is uniform, and a user adjusts the output every time, or does not adjust the output at all.

### Solution to Problem

To solve the problems described above, the present invention provides a brush which is capable of applying an electric current to a human body and includes a brush body, electrodes respectively provided at tips of brush bristles, a sensor provided on a surface of the brush body on which the brush bristles are provided, and a controller configured to control motions of the brush bristles based on a detection output signal of the sensor.

### Advantageous Effects of Invention

According to the present invention, the sensor detects the conditions of the scalp, and the brush performs electric stimulation by applying a current of a specific frequency, or performs ion introduction/lead-out by applying a weak current, so as to be suitable for the conditions of the scalp of a target portion, resulting in exhibiting electric effects or ion effects to the maximum without waste.

### BRIEF DESCRIPTION OF DRAWINGS

FIG. 1 shows the brush according to the present invention, where (a) illustrates a perspective view from the side opposite to the side where brush bristles are provided, and (b) illustrates a perspective view from the side where the brush bristles are provided.

FIG. 2 shows the brush according to the present invention, where (a) illustrates a view from the side where the brush bristles are provided, (b) illustrates a view from the side of (a), and (c) illustrates a view from the back side of (a).

FIG. 3 shows a stand of the brush according to the present invention, where (a) illustrates a perspective view, and (b) illustrates a view from above in the direction of brush insertion.

FIG. 4 shows the stand of the brush according to the present invention, where (a) illustrates a view from the front, and (b) illustrates a view from the rear.

FIG. 5 shows the stand of the brush according to the present invention, where (a) illustrates a side view, and (b) illustrates other side view.

### DESCRIPTION OF EMBODIMENTS

The present invention provides the configuration including a brush body, electrodes respectively provided at the tips of brush bristles, a sensor provided on the surface of the brush body on which the brush bristles are provided, and a controller configured to control the motions of the brush bristles on the basis of a detection output signal of the sensor, in order to solve the problem in which, due to uniform output in spite of ununiform scalp and hair conditions in the entire cephalic region, a user adjusts the output every time.

The sensor may use various types of electronic and electric elements and techniques for grasping the conditions of the hair and scalp. In an example, the sensor may detect electrical resistance of the scalp between two contact points by contacting with the scalp at two points, may detect the degree of dryness by contacting on the basis of electrical resistance or electrostatic capacitance, may detect so-called dandruff and dust including metabolite, sebum, shampoo residue and mold by contacting with the scalp by use of image processing technology, and may detect the stiffness (softness) of the scalp by pressing against the scalp.

In an example, the controller may control a voltage in order to keep an output current value constant with respect to a fluctuating resistance value, on the basis of the detection output signal of the sensor described above, and may control the output patterns through the electrodes respectively provided to the brush bristles.

This allows a user to perform scalp care constantly with the optimum output to the conditions of target portions, simply by turning on the power and performing normal brushing without the need of arbitrary setting for respective portions.

### EXAMPLE

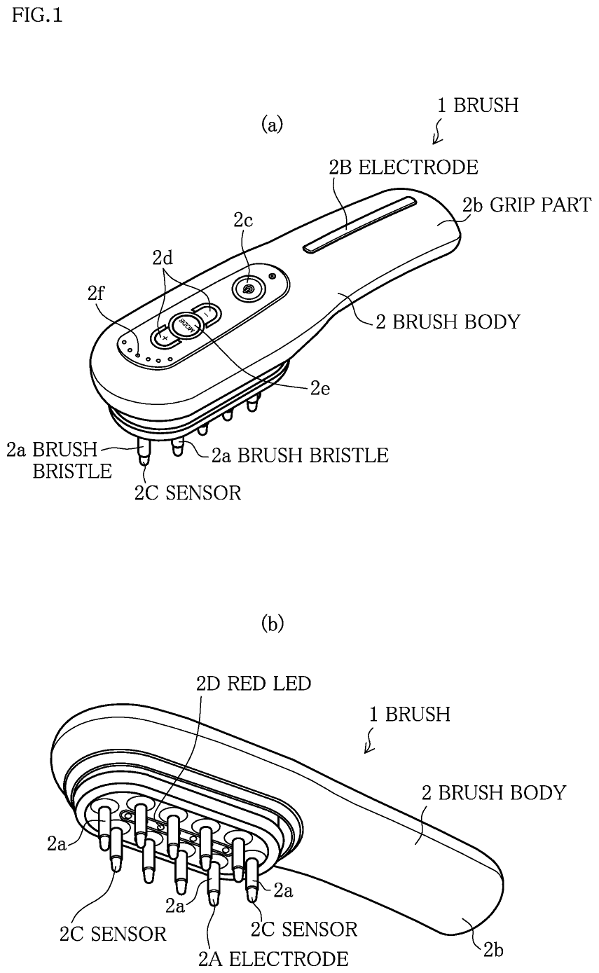

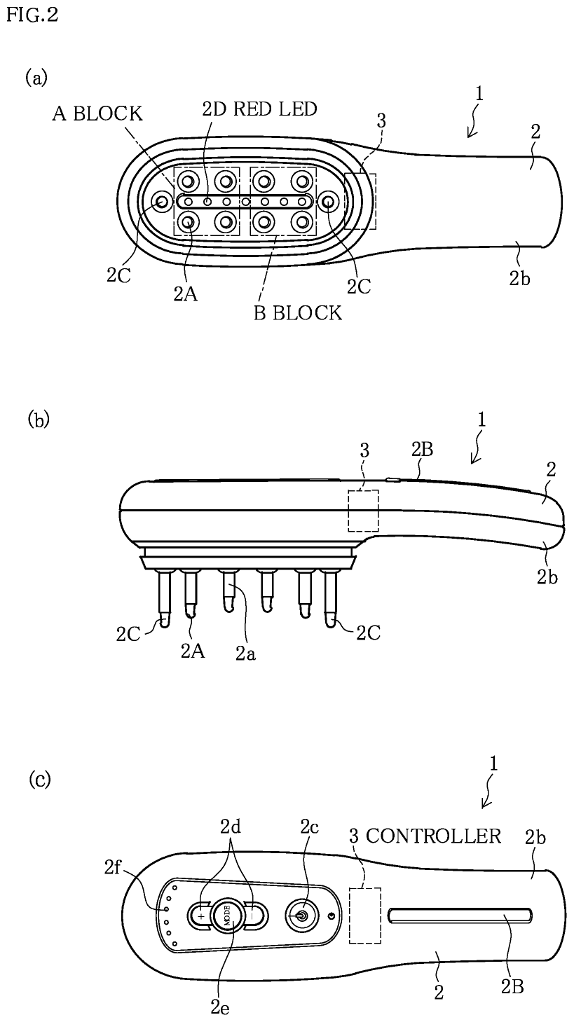

Hereinafter, the brush according to the present invention will be described with reference to the drawings. As shown in FIG. 1 and FIG. 2, a brush 1 in the present example according to the present invention includes a brush body 2, electrodes 2A provided at the tips of brush bristles 2a, a sensor 2C provided on the surface of the brush body 2 on which the brush bristles 2a are provided, and a controller 3 configured to control the motions of the brush bristles 2a on the basis of the detection output signal of the sensor 2C, 2C.

The brush body 2 is provided with an electrode 2B in a grip part 2b of the brush body 2 on the back surface of the surface on which the brush bristles 2a are provided (hereinafter, referred to as a brush face). When the electrodes 2A provided at the tips of the brush bristles 2a are brought into contact with the human body via the scalp, and the electrode 2B provided in the grip part 2b is brought into contact with the gripping hand, a circuit via the human body is formed, and is supplied with a predetermined current through the electrodes 2A.

The brush body 2 is provided with a power switch 2c, a volume up/down switch 2d, a mode change switch 2e and a volume indicator 2f, on the back surface of the brush face. The power switch 2c is configured to turn on/off the operation of the brush 1 according to the present invention, as a whole. The volume up/down switch 2d is configured to increase and decrease the output, as a whole.

In the present example, the mode change switch 2e is configured to switch a mode among an interference wave output mode, an ion introduction/lead-out mode, and an electroporation mode, which will be described later. The volume indicator 2f is configured to indicate an output volume.

In the present example, each of the brush bristles 2a (electrodes 2A) is provided with a cushioning mechanism not illustrated inside the brush face, and configured to be sunk at the portion where the shape of the head protrudes. Therefore, the brush bristles 2a are kept in constant contact with the scalp. In the case of the present example, each of the brush bristles 2a is provided not only with the cushioning mechanism, but also with a mechanism for micro-vibration (forward/rear motion, left/right up/down motion, circular motion).

On the brush face, ten brush bristles 2a in total are arranged, including four bristles in the longitudinal direction of the brush body 2, two bristles (rows) in the short direction, and two bristles at the both ends in the longitudinal direction. A plurality of red LEDs 2D which are expected to give a promoting effect on hair growth are arranged between the two rows in the short direction. In the present example, there are two blocks of a block A and a block B each which includes four brush bristles 2a (electrodes 2A) and is surrounded by a dash-and-dot line, and an interference frequency current flows in each of the block A and the block B.

Further, in the present example, the brush bristles 2a arranged at the both ends in the longitudinal direction of the brush body 2 on the brush face are provided with the sensors 2C, 2C at the tips thereof, respectively. Each of the sensors 2C, 2C is also provided with the cushioning mechanism not illustrated inside the brush face, and is kept in constant contact with the scalp.

The controller 3, which is provided inside the brush body 2, is electrically connected directly or indirectly to the sensors 2C, 2C, the electrodes 2A, the electrode 2B, the red LEDs 2D, the power switch 2c, the volume up/down switch 2d, the mode change switch 2e, and the volume indicator 2f. The controller 3 is provided with a ROM (not illustrated) and a chip which store the operation program of the brush 1 according to the present invention and the database corresponding to output of respective modes based on the detection results of the sensors 2C, 2C, on the same substrate.

As described above, the brush 1 having the above configuration in the present example according to the present invention has a massage mode, an interference wave output mode, an ion introduction/lead-out mode, and an electroporation mode. When a mode is changed with the mode change switch 2e, and the first output is adjusted with the volume up/down switch 2d (if the first output is not adjusted, the output is set to an intermediate volume), the controller 3 performs overall control to ensure appropriate effects without excess or deficiency for respective portion according to the scalp conditions obtained by the sensors 2C, 2C.

In the massage mode, the impedance of the scalp between the sensors 2C, 2C is detected, and the controller 3, when determining that the scalp is stiff on the basis of the impedance, makes the brush bristles 2a of the portion vibrate to perform scalp massage. The strength of the vibration is also programmed in advance in the table based on the impedances described above of the scalp.

In the interference wave output mode, the impedance of the scalp between the sensors 2C, 2C is detected, and the frequency differences (interference frequencies) of the current to be applied through the electrodes 2A, and the method of causing interference are adjusted on the basis of the impedance above, in the block A and the block B described above of the electrodes 2A. The method of causing interference herein means the method of applying a current alternately to the electrodes 2A disposed opposite to each other (in pair), the method of repeatedly applying a current to be on/off by use of all the two pairs of the electrodes 2A, 2A, 2A, 2A, or other method.

In the ion introduction/lead-out mode, the impedance of the scalp between the sensors 2C, 2C is detected, and a positive voltage of a weak current (10 μA to 5 mA) to the electrode 2A corresponding to a positive pole or all the electrodes 2A in the block A and the block B described above of the electrodes is adjusted on the basis of the impedance, that is, on the basis of the degree of dirtiness, so that old keratin, dust, dead mites and the like negatively charged are attracted to the brush bristles 2a.

In the ion introduction/lead-out mode, the impedance of the scalp between the sensors 2C, 2C is detected, and a negative voltage of a weak current (10 μA to 5 mA) to the electrode 2A corresponding to a negative pole or all the electrodes 2A in the block A and the block B described above of the electrodes is adjusted on the basis of the impedance, so that lotion, hair restorer, scalp conditioning agent or the like negatively charged is made to penetrate into the scalp, by utilizing the principle of like charges repulsion.

In the electroporation mode, the impedance of the scalp between the sensors 2C, 2C is detected, and on the basis of the impedance, a nutrient composition for scalp such as collagen, hyaluronic acid, or placenta is made to penetrate into the skin of the portion to be subjected to the penetration, by applying a voltage (−100V to −50V) to the portion and creating gaps between skin cells.

All the red LEDs 2D are lighted when the power switch 2c is on. Alternatively, the red LEDs 2D may be individually lighted with varying luminosity on the basis of the detected impedance of the scalp between the sensors 2C, 2C.

In other words, in the present example, elements such as so-called stiffness, bloodstream and dirtiness of the scalp are detected as the impedances between the sensors 2C, 2C, and the controller 3 controls a current value, a voltage value, and a frequency appropriately and consistently without excess or deficiency according to the detection results of the impedance. This allows to perform electric stimulation or ion introduction/lead-out suitable for the conditions of the scalp of a target portion, resulting in exhibiting such effects to the maximum.

In the example described above, the brush 1 according to the present invention is used for the cephalic region. The brush 1 may be used for face, as an example. In this case, the controller 3 calculates the ratio of fat and muscle under the skin of face on the basis of the detected impedance between the sensors 2C, 2C. One of or both of the sensors 2C, 2C measure the degree of sinking (load) of the sensor 2C or the sensors 2C, 2C into the brush face when the brush 1 is lightly pressed onto the skin, and output the result to the controller 3. The controller 3 converts the result to the degree of flabbiness.

In the case of the face having a lot of subcutaneous fat and flabby fat, the controller 3 controls and makes the brush bristles 2a vibrate toward a subcutaneous fat layer to remove the subcutaneous fat, on the basis of the balance between the subcutaneous fat and the muscle of the face obtained by the sensors 2C, 2C and the table with respect to the degree of flabbiness.

In the case of the face having low subcutaneous fat and stiffened muscle, the controller 3, on the basis of the balance between the subcutaneous fat and the muscle of the face obtained by the sensors 2C, 2C and the table with respect to the degree of flabbiness, controls and applies an electric current of a specific frequency through the electrodes 2A of the brush bristles 2a, or makes the frequencies of the currents flowing through the plurality of electrodes 2A interfere with each other, to massage and activate the muscle.

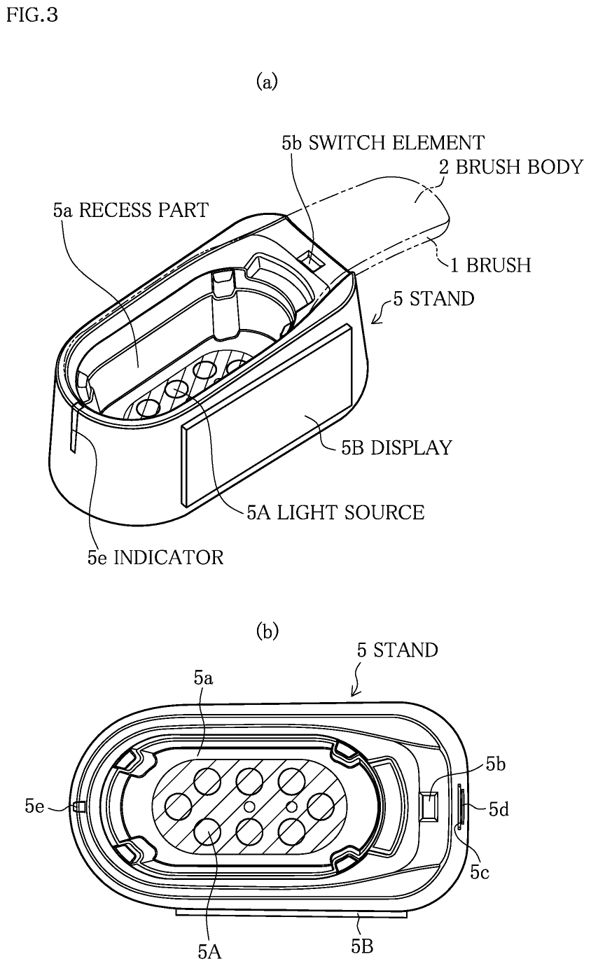

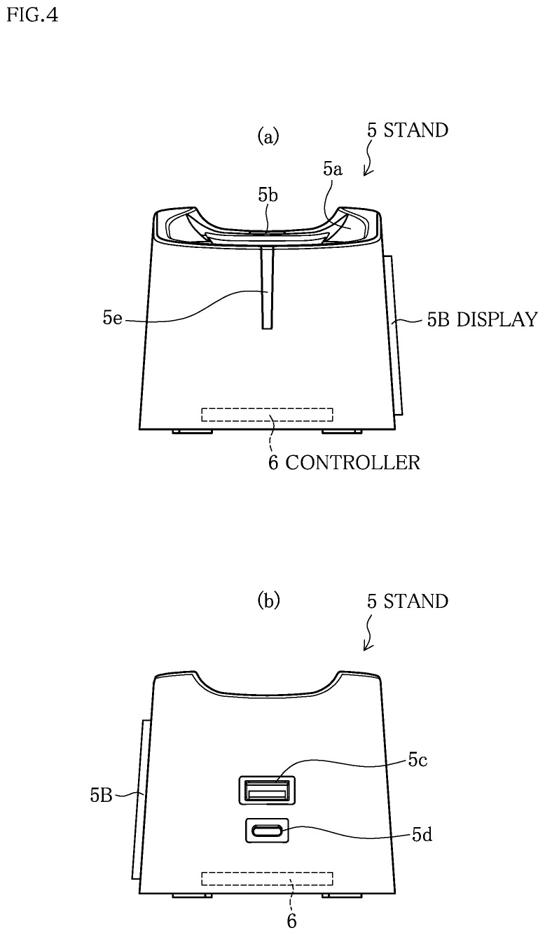

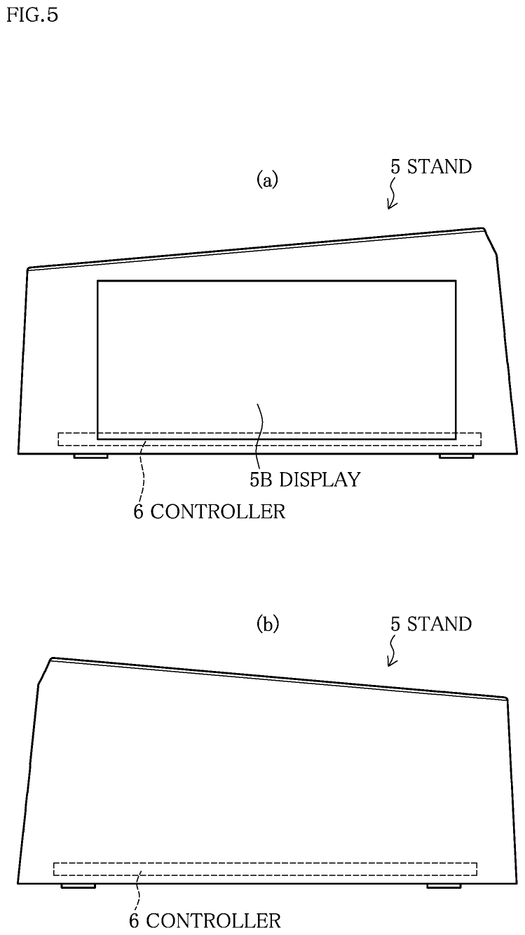

As shown in FIG. 3 to FIG. 5, the brush according to the present invention may include a stand. The configuration of the brush 1 with a stand 5, which will be described below, may be the same as the configuration of the example described above, except that the brush 1 communicates with the stand 5 wirelessly and is connected by wire for charging.

The stand 5 has a recess part 5a with the upper face opened, and the brush bristles 2a and the sensors 2C, 2C of the brush 1 are stored in the recess part 5a. The recess part includes light sources 5A configured to emit UVA (ultraviolet A) and UVC (ultraviolet C) in a hatched area on the inner bottom face, as an example.

The stand 5 has a switch element 5b in the rear part. In the present example, the switch element 5b is configured to detect whether the handle of the brush 1 is in contact therewith. When the contact of the handle of the brush 1 is detected, the light sources described above are turned on to sterilize the brush face, the brush bristles 2a, and the sensor 2C, 2C. When the detachment of the handle of the brush 1 from the switch element 5b is detected, at least the light source 5A which emits UVC out of the light sources 5A described above is immediately turned off.

In the present example, the stand 5 is provided with a display 5B allowing touch operation, on the right side, as an example. The display 5B allows monitoring of various types of information, and further allows remote operation to the brush 1.

The stand 5 incorporates a controller 6 configured to control the stand 5 itself and perform interlocking control with the brush 1. The controller 6 is connected to the light sources 5A, the display 5B, a communication device and the like, and provided with a ROM storing a predetermined program and the like on the same substrate.

The stand 5 is provided with a terminal 5c for connecting the brush 1 by wire, and a power supply terminal 5d for connecting a power cord connected to an outlet not illustrated, on the rear end face. The stand 5 is provided with an indicator 5e which indicates a state of charging to the brush 1 or a power storage amount by color or blinking pattern, on the front end face.

The brush 1 according to the present invention including the stand 5 may operate, as follows. In the present example, the brush 1 is attached to the stand 5, and the power cord connected to the brush 1 as described above is connected to the terminal 5c, so that the brush 1 is charged. The power cord is connected between the power supply terminal 5d and the outlet, so that power is supplied to the stand 5. Since the brush 1 is attached to the stand 5, the switch element 5b detects the contact of the handle of the brush 1, and the light sources 5A emit UVA and UVC.

When the brush 1 is disconnected from the power cord and lifted from the stand 5, the switch element 5b detects the disconnection of the handle of the brush 1, and then the controller 6 turns off light sources 5A. Thereafter, the brush 1 is turned on, and the brush 1 performs the operation described above. In the present example, the stand 5 separately displays head portions and face portions according to a scalp mode or a face mode, along with the operation described above of the brush 1, on the display 5B.

The data detected or measured by the sensor 2C, 2C is output by the brush 1 to the stand 5, and the controller 6 of the stand 5 converts the received data, and outputs it to the display 5B. In the case where the brush 1 is used for the scalp, the controller 6 displays dirtiness, stiffness and the like of the scalp, in a different way.

This allows a user to use the brush 1 by visually checking the conditions of a use portion. In an example, prior to the actual operation of the brush bristles 2a, a user makes only the sensor 2C, 2C operate just once to grasp the conditions of the use portion before the use of the brush 1. Thereafter, the user can confirm the effects by the use of the brush 1, by checking the display 5B during the use or after the use of the brush 1.

The moving of the brush 1 during the use of the brush 1 is displayed as a trace on the display 5B. This allows a user to determine whether to move the brush 1 to a target portion, or whether to use the brush 1 intensively at one portion without moving, allowing more appropriate use of the brush 1.

The display 5B, which allows touch operation in the present example, further allows optional operation such as of indicating a more concrete numerical value, finely adjusting the strength of the motion of the brush 1 at a target portion, or making the red LEDs 2D arbitrarily emit light not according to a program, by, for example, touching of a dirty portion on the display 5B. It is noted that with respect to such optional operation, a completely unrelated and unsafe operation is programmed so as not to be displayed as an option.

The brush 1 described above may incorporate, for example, a microscope to display imaging conditions on the display 5B of the stand 5. The stand 5 or the brush 1 may include storage means so as to display use history on the display 5B.

The program of the stand 5 may include maintenance modes of the brush 1 and the stand 5, thereby allowing to notify a user of cleaning according to use time, failure points and the like.

### REFERENCE SIGNS LIST

1: BRUSH
2: BRUSH BODY
2A: ELECTRODE
2B: ELECTRODE
2D: RED LED
3: CONTROLLER
5: STAND
5A: LIGHT SOURCE
5B: DISPLAY
6: CONTROLLER

1. A brush configured to apply an electric current to a human body, the brush comprising:
a brush body;
electrodes respectively provided at tips of brush bristles;
a sensor provided on a surface of the brush body, the surface with the brush bristles provided; and
a controller configured to control motions of the brush bristles based on a detection output signal of the sensor.

2. The brush according to claim 1, wherein
a cushioning mechanism for the brush bristles is provided inside the surface with the brush bristles provided.

3. The brush according to claim 1, wherein
a stand configured to store the brush bristles of the brush body while keeping a predetermined posture of the brush bristles facing toward a mounting surface.

4. The brush according to claim 2, wherein
a stand configured to store the brush bristles of the brush body while keeping a predetermined posture of the brush bristles facing toward a mounting surface.
